(19) United States
(12) Patent Application Publication
(10) Pub. No.: US 2021/0393383 A1
(43) Pub. Date: Dec. 23, 2021

(54) Compact extraoral suction passage tip

(71) Applicants: James R. Bullinger, Fork, MD (US); Sean T. Zakielarz, Fallston, MD (US)

(72) Inventors: James R. Bullinger, Fork, MD (US); Sean T. Zakielarz, Fallston, MD (US)

(21) Appl. No.: 17/354,754

(22) Filed: Jun. 22, 2021

Related U.S. Application Data
(60) Provisional application No. 63/042,041, filed on Jun. 22, 2020.

(51) Int. Cl. A61C 17/08; A61C 17/06; A61C 17/10; A61C 17/12

(52) U.S. Cl. CPC A61C 17/08; A61C 17/12; A61C 17/10; A61C 17/092

(57) ABSTRACT

A compact extraoral suction passage tip to help protect a dentist from infectious materials. The compact extraoral suction passage tip is made of an internal suction passage. The proximal end can be slid into a High Volume Evacuator (HVE) valve or other suctioning device, and the distal end can be placed near a patient's mouth to collect infectious materials and placed in a position that will not significantly impede dental operations. The distal end is shaped so that it can lie on or near the upper portion of a patient's bottom lip to provide suction capability. One embodiment contains ridges to help patients know where to hold the compact extraoral suction passage tip.

### CROSS-REFERENCE TO RELATED APPLICATIONS

This application claims priority from U.S. Provisional Application No. 63/042,041 filed on Jun. 22, 2020, which is hereby entirely incorporated by reference.

### BACKGROUND OF THE INVENTION

The present invention pertains to the area of an extraoral suction passage tip for placement of vent slot opening above or resting on the top of a patient's bottom lip for use in dentistry with said vent slot opening pointing towards the entrance of said patient's oral cavity.

Applicable patent classifications include the following:
CPC Classification: A61C 17/06: Saliva removers; Accessories therefor
U.S. Classification: 433/91: Dentistry apparatus, Having suction orifice

The following is a listing of reference material.

### U.S. Patents

| | | | Current | |
|---|---|---|---|---|
| Pat. No. | Appl. No. | Issue Date | U.S. Class | Patentee |
| 7,261,560 | 10/999,008 | Aug. 28, | 433/95; | Abo |
| | | 2007 | 433/93; | |
| | | | 433/96; | |
| | | | 433/94; | |
| | | | 433/91 | |
| 5,762,496 | 08/520,956 | Jun. 9, 1998 | 433/93; | Albtertson et al. |
| | | | 433/94 | |
| 6,186,783 | 08/953,108 | Feb. 13, | 433/91; | Brassil et al. |
| | | 2001 | 433/96 | |
| 5,407,353 | 08/087,755 | Apr. 18, 1995 | 433/93; | Clementz |
| | | | 433/136 | |
| 5,688,121 | 08/547,028 | Nov. 18, | 433/96; | Davis et al. |
| | | 1997 | 433/91 | |
| 5,882,194 | 692917 | Mar. 16, | 433/29; | Davis et al. |
| | | 1999 | 433/91; | |
| | | | 433/95 | |
| 7,845,944 | 11/416,223 | Dec. 7, | 433/91 | DiGasbarro |
| | | 2010 | | |
| 5,066,228 | 07/571,182 | Nov. 19, | 433/91; | Doundoulakis et |
| | | 1991 | 433/95 | al. |
| 5,152,686 | 07/691,074 | Oct. 6, | 433/93; | Duggan et al. |
| | | 1992 | 433/140; | |
| | | | 433/29 | |
| 6,309,218 | 09/712,520 | Oct. 30, | 433/93; | Ellenbecker |
| | | 2001 | 433/138; | |
| | | | 600/238 | |
| 8,545,401 | 12/925,866 | Oct. 1, | 600/205; | Hajarian et al. |
| | | 2013 | 433/91 | |
| 8,734,152 | 13/099,224 | May 27, 2014 | 433/93 | Hirsh et al. |
| 9,888,989 | 14/779,745 | Feb. 13, | 1/1 | Ishizaki et al. |
| | | 20181 | | |
| 4,017,975 | 05/669,244 | Apr. 19, 1977 | 433/94; | Johnson |
| | | | 433/91 | |
| 4,058,896 | 05/649,211 | Nov. 22, | 433/91 | Moore |
| | | 1977 | | |
| 8,360,773 | 13/066,573 | Jan. 29, | 433/96; | Pasha |
| | | 2013 | 604/902 | |
| 7,300,401 | 11/091,026 | Nov. 27, | 600/238; | Patrickus |
| | | 2007 | 600/242; | |
| | | | 433/93 | |
| 8,870,568 | 11/374,775 | Oct. 28, | 433/93 | Ream |
| | | 2014 | | |
| 10,136,975 | 15/265,762 | Nov. 27, | 1/1 | Reyes |
| | | 2018 | | |
| 5,490,780 | 08/219,345 | Feb. 13, | 433/93; | Riewenherm |
| | | 1996 | 433/31 | |
| 5,127,411 | 07/725,197 | Jul. 7, 1992 | 128/863; | Schoolman |
| | | | 128/917; | |
| | | | 433/91; | |
| | | | D24/110.4 | |
| 5,441,410 | 08/039,162 | Aug. 15, | 433/93; | Segerdal |
| | | 1995 | D24/176; | |
| | | | D24/112; | |
| | | | 433/95; | |
| | | | 433/96 | |
| 4,325,695 | 06/196,981 | Apr. 20, 1982 | 433/91; | Sundelin et al. |
| | | | 433/94 | |
| 5,437,651 | 08/116,201 | Aug. 1, 1995 | 604/313; | Todd et al. |
| | | | 15/420; | |
| | | | 604/317; | |
| | | | 604/323; | |
| | | | 604/902 | |
| 6,585,512 | 09/982,651 | Jul. 1, 2003 | 433/91 | Van Hale |
| 5,114,342 | 07/607,570 | May 19, 1992 | 433/95; | Young et al. |
| | | | 137/512.4; | |
| | | | 604/119 | |

### U.S. Patent Application Publications

| Publication Number | Publication Date | Applicant |
|---|---|---|
| 2003/0203336 A1 | Oct. 30, 2003 | Somodi |

### Nonpatent Literature Documents

Emmons, Laura and Cheri Wu, R D H, “High-volume evacuation: Aerosols—It's What You Can't See that Can Hurt You” (Jul. 1, 2017), https://www.rdhmag_com/patient-care/article/16409779/highvolume-evacuation-aerosolsits-what-you-cant-see-that-can-hurt-you.
Harrel, Stephen K. and John Molinari, American Dental Association, JADA, Vol. 135, “Aerosols and Splatter in Dentistry” (April 2004) https://jada.ada.org/articie/S0002-8177(14)61227-7/pdf.
Jacks, Mary E. J Dent Hyg. 76(3):202-6 “Laboratory Comparison of Evacuation Devices on Aerosol Reduction” (Summer 2002), https.//pubmed.ncbi.nlm.nih.gov/12271865/.
Tekitronics, “ADS: Extraoral Dental Suction System Droplets and Aerosols Terminator” https://tekitronics.com/products/ajax-dent/.

### BACKGROUND

Dental aerosols, splatter, and droplets occur during dental procedures. If contaminated, these can spread disease through the air and surfaces. According to Stephen K. Harrel, D.D.S. and John Molinari, PhD, “[t]he smaller particles of an aerosol have the potential to penetrate and lodge in the smaller passages of the lungs and are thought to carry the greatest potential for transmitting infections” (Harrel, 2). With the COVID-19 epidemic, other future pandemics/epidemics, and flu seasons, dentist offices could be concerned about performing dental work on patients because of fear of becoming infected from these airborne particulates.

Suction devices to help control aerosol can be generally categorized as intraoral and extraoral. Intraoral suction devices are placed within the oral cavity to help reduce aerosols from escaping. Extraoral suction devices are placed outside the oral cavity, typically using devices with fluid dynamic properties to suction aerosol cloud particulates away from patients. These extraoral suction devices are usually connected to an external central vacuum system through high-volume evacuator (HVE).

Saliva suction devices are common intraoral suction devices, but they do not protect against the spread of disease through airborne particulates (Emmons). Examples of such saliva suction devices include Abo, Albertson, both Davis patents, DiGasbarro, Doundoulakis, Johnson, Moore, Segerdal, Todd, and Young.

There are several intraoral evacuation devices marketed to decrease airborne particulates associated with dental operations, but they contain several drawbacks. One big drawback of intraoral devices is that they take up space within the oral cavity, reducing the amount of space a dentist has to perform dental work. Also, if there is no easy way for the intraoral device to stay in place within a patient's oral cavity, then a separate person may have to hold the device so that it does not interfere with dental procedures. Several solutions to this problem have been proposed, but they usually still make it more cumbersome to perform dental operations, as they are within the oral cavity, which is the same location in which the dental operations are being performed (see Albtertson, Clementz, Ream, Somodi, Sundelin). Some of these solutions can be uncomfortable for patients and some of the solutions attach to various parts of the oral cavity to hold the intraoral device in place. For example, in Clementz the intraoral device is held in place using an adjacent tooth. Such solutions to hold the intraoral devices could presumably cause more patient discomfort during dental procedures. Examples of evacuation devices that have the general disadvantages associated with intraoral evacuation devices include Brassil, Duggan, Ellenbecker, Hirsh, Johnson, Reyes, Riewenherm, Sundelin, Pasha, Patrickus, and Van Hale.

Other extraoral devices are primarily hoods or masks that are attached to evacuation systems. With these types of extraoral evacuation systems, dental operators have to work around bulky hoods or masks that block significant access to the patient's oral cavity (see Ishizaki and Tekitronics for examples of extraoral hood devices and Brassil for an extraoral mask with the same access problem). Some extraoral evacuation systems do not connect to an external central vacuum system through HVE, which reduce the amount of force available or the ability to effectively contain aerosols.

Another extraoral evacuation device lined the entire outer part of the mouth or lips (see Schoolman). Having an extraoral evacuation device cover the entire circumference of a patient's mouth could be uncomfortable for the patient, and it could be difficult for dental operators to work around.

### BRIEF SUMMARY OF THE INVENTION

An embodiment of the present invention comprises a compact extraoral suction passage tip with a vent slot opening with a different shape than the hollow cylindrical tube that it is connected to on the distal end of said compact extraoral suction passage tip. On the proximal end of the compact extraoral suction passage tip, a hollow cylindrical tube can be operably connected to an external central vacuum system through HVE by sliding the hollow cylindrical tube into an external HVE valve on the proximal end of the hollow cylindrical tube with a suction passage formed between the distal and proximal ends of the compact extraoral suction passage tip. The preferred embodiment (i.e., best mode) comprises that shown in FIGS. 1A, 1B, 1C, and 1D with semicircular ridges to allow a patient to know where the compact extraoral suction passage tip should be placed on his or her face, which is in a position wherein the vent slot opening is on or around the top of said patient's bottom lip with the vent slot opening pointing towards the entrance to said patient's mouth. In the preferred embodiment, the compact extraoral section passage tip is preferred to be made of High Density Polyethylene (HDPE) that can be discarded. In the preferred embodiment, the semicircular ridges are intended to be placed around a patient's chin, which brings the vent slot opening just on or around the top of patient's bottom lip and pointed towards the entrance of the oral cavity.

### Advantages

Several advantages of one or more aspects of the compact extraoral passage tip embodiments are as follows:
The embodiments do not significantly impede dental operators from having full access to the oral cavity to perform dental procedures. Unlike other extraoral devices, the embodiments are not in the shape of a hood or mask that would substantially impede a dentist from accessing the oral cavity. The embodiments also do not surround the entire oral cavity.
The embodiments are not inserted in the oral cavity, so they do not impact the space in the oral cavity opening available for operations, and they fix the patient discomfort issue associated with having a suction device inside an oral cavity.
The embodiments can be manufactured at a price that would allow them to be disposable, which decreases chances of infection associated with reusable devices that may not be properly autoclaved between patients. Embodiments can also be manufactured to be autoclavable.
The embodiments can be attached to an external central vacuum system through HVE. HVE systems are installed in most dentist's offices already, which will reduce the price-point of the compact extraoral suction passage tip.
The embodiments have a lower suction strength than that which would be expected from placing an HVE tube near the entrance of a patient's oral cavity because of the embodiments' fluid dynamic properties. This allows the embodiments to be used near or on the upper portion of a patient's bottom lip right outside of the entrance of the patient's oral cavity with the vent slot opening pointing towards the entrance to the patient's oral cavity with little to no discomfort.

### BRIEF DESCRIPTION OF THE DRAWINGS

FIG. 1A shows a top view of the preferred embodiment.

FIG. 1B shows a front view of the preferred embodiment.

FIG. 1C shows an isometric view of the preferred embodiment.

FIG. 1D shows a side view of the preferred embodiment.

FIG. 2A shows the top view of one embodiment.

FIG. 2B shows a front view of the one embodiment with a sample width dimension of the vent slot opening, which can vary across embodiments.

FIG. 2C shows an isometric view of one embodiment.

FIG. 2D shows a side view of one embodiment with sample dimensions, which can vary across embodiments,

FIG. 3 shows one embodiment as it could be positioned on a patient with said patient holding the embodiment in place by holding the external HVE valve.

### DETAILED DESCRIPTION OF THE INVENTION

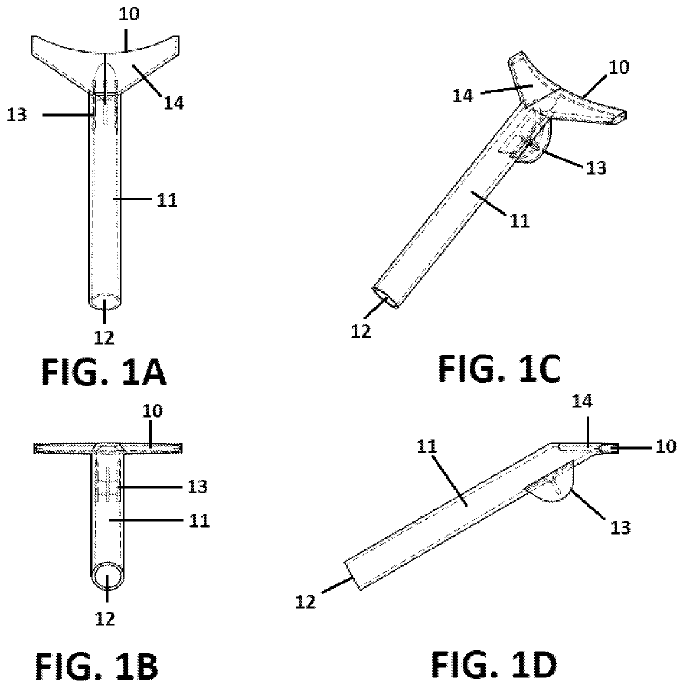

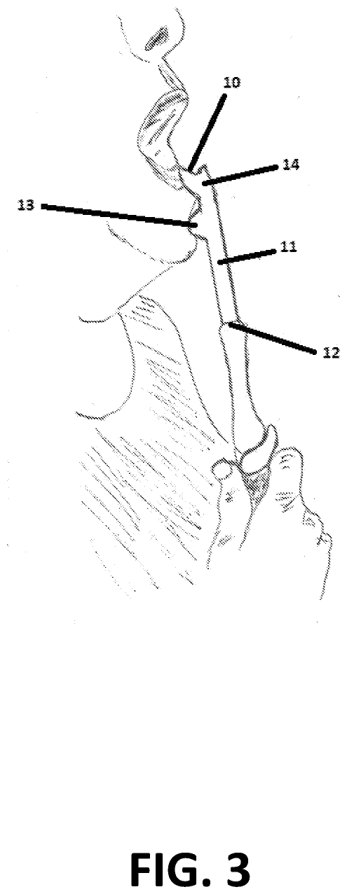

The preferred embodiment of the compact extraoral suction passage tip is illustrated in different views in FIGS. 1A, 1B, 1C, and 1D. An embodiment is shown as in its preferred placement on a patient in FIG. 3. In any embodiment, the compact extraoral section passage tip is preferred to be made of High Density Polyethylene (HDPE) that can be discarded. However, other embodiments can also be made of Polypropylene (PP) medical grade, Acetal Copolymer (POM-C), or other resilient of semi-resilient material.

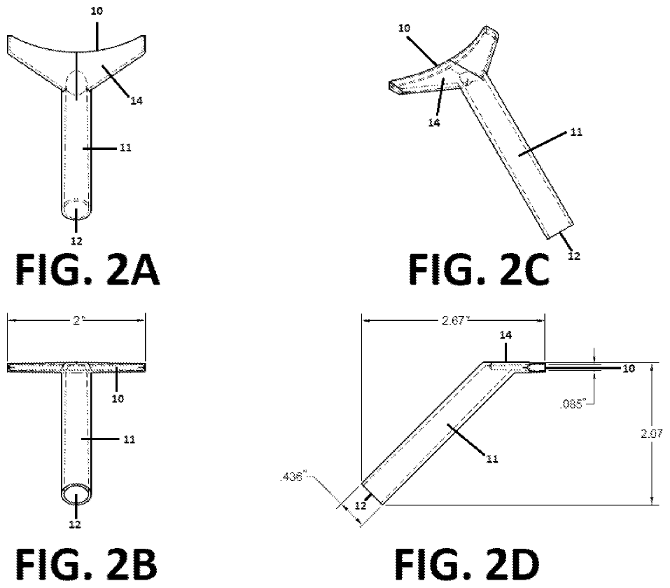

At the distal end of the compact extraoral suction passage tip in FIGS. 1A, 1B, 1C, 1D, 2A, 2B, 2C, 2D, and 3, there is vent slot opening 10, into which aerosols and splatter from a patient's oral cavity during a procedure flow into. The aerosols and splatter then flow through the compact extraoral suction passage tip toward the proximal end of the the compact extraoral suction passage tip. The contaminates then flow into an external central vacuum system through external HVE. In the displayed embodiments, the vent slot opening 10 is a roughly rectangular slot. In the embodiments shown in FIGS. 1A, 1B, 1C, 1D, 2A, 2B, 2C, 2D, and 3, a wing-shaped collector 14 contains said vent slot opening 10 at said wing-shaped collector's distal end and connects to hollow cylindrical tube 11 at its proximal end.

In the preferred embodiment, wing-shaped collector 14, which contains vent slot opening 10 at its distal end, is of the same type of material as hollow cylindrical tube 11 and consists of one continuous piece of material. Alternatively, wing-shaped collector 14 with vent slot opening 10 could be made of the same or different material than hollow cylindrical tube 11 and could be created as separate pieces or a singular continuous piece. In either case, the continuous piece or the separate pieces will be manufactured using injection molding.

In the preferred embodiment in FIGS. 1A, 1B, 1C, and 1D, the hollow cylindrical tube 11 contains three beveled semicircular ridges 13 near the wing-shaped collector 14. In the preferred embodiment, the beveled ridges 13 are designed to be placed on or around a patient's chin. In the preferred embodiment, the beveled ridges 13 are part of the same continuous piece of material as the rest of the compact extraoral suction passage tip. However, other embodiments can allow for beveled ridges that attach to the hollow cylindrical tube as a separate piece and other embodiments can have a single or plurality of beveled semicircular ridges. In other embodiments the beveled semicircular ridge or ridges could be placed in another appropriate position on the patient's face to help the patient know where to hold the compact extraoral suction passage tip in place.

In other embodiments, friction-inducing mechanisms between the embodiments and various body parts of a patient could be included.

In the preferred embodiment, at the proximal end of the compact extraoral suction passage tip, a cylindrical tube opening 12 has a circular cross-section opening to the hollow cylindrical tube 11. Said hollow cylindrical tube 11 and cylindrical tube opening 12 are shaped so that they can be slipped into a standard external HVE valve. The external HVE valve is connected, on its other end, to an external central vacuum system. In other embodiments, the cylindrical tube opening 12 can be a shape that would accommodate other relevant vacuum tubes.

In the preferred embodiment, fluid flows from vent slot opening 10 through the wing-shaped collector 14, then into cylindrical tube opening 12, and then to the external central vacuum system via external HVE. In the preferred embodiment, the proximal end of the inner and outer surfaces of hollow cylindrical tube 11, will be of the correct diameters so that someone can slide the proximal end of the hollow cylindrical tube 11 into an external HVE valve that is connected to an external central vacuum system.

In all embodiments, the proximal end of hollow cylindrical tube 11 is connected to an external HVE line or other evacuator/vacuum source. In the preferred embodiment, vent slot opening 10 is placed near the patient's oral cavity near or along the top of said patient's bottom lip with vent slot opening 10 pointing toward the entrance to the patient's oral cavity. FIG. 3 shows the vent slot opening 10 being placed along the upper portion of the patient's bottom lip pointing into the entrance of the patient's oral cavity. However, the vent slot opening 10 can be molded to fit in other areas directly outside the patient's oral cavity.

The various embodiments can be held in place by the patient or dental office worker. If the embodiment contains friction-inducing components, it is possible that the compact extraoral suction passage tip will stay in place without the need for anyone to hold the embodiment in place.

There are various possibilities with respect to the positioning, shape, dimensions, dimension ratios, and size of vent slot opening 10 and wing-shaped collector 14. For instance, it can be easily molded to fit somewhere else along the oral cavity. Also, further testing may reveal that having the vent slot opening molded to another area of the face may do a better job at reducing airborne aerosols during dental operations. Other embodiments may include rectangular, ellipsis, or other shapes for the vent slot opening.

In the preferred embodiment, a wing-shaped collector 14 transforms the hollow cylinder shape of hollow cylindrical tube 11 to a rectangular vent slot opening 10 by widening the passage along the horizontal axis and narrowing the passage along the vertical axis as it transverses from the hollow cylindrical tube 11 towards the vent slot opening 10. In the preferred embodiment, the angle along the axial axis between the wing-shaped collector 14 and the hollow cylindrical tube 11 are at an angle of approximately 45 degrees as viewed from the side (FIG. 1D).

Materials of 10, 11, 12, 13, and 14 may also be modified as needed. Specifically, there could be an option to use disposable or autoclavable materials in manufacturing to allow for reuse, as one or the other may be desired by some dentist offices.

Various friction-generating means can be added along vent slot opening 10 and hollow cylindrical tube 11 such as various ridges, groves, bumps, etc. to help keep the compact extraoral suction passage tip in place.

Various ways to enable a patient to wear the compact extraoral suction passage tip could allow for alternative positions and potentially advantageous ways to ensure that the compact extraoral suction passage tip does not move during dental operations.

Variations on the pressure/fluid flow by changing ratios in the various parts of the compact extraoral suction passage tip are also possible.

### Advantages of Additional/Alternative Embodiments

Cases may arise when a patient has some type of deformity or needs a special type of operation that would make using the vent slot opening 10 on or near the top of the bottom lip and pointing towards the entrance of the oral cavity less optimal than other locations around the outside of the oral cavity. Having the ability to vary the positioning, molding, and the shape of the vent slot opening 10 would help in these situations. Other options for placing or wearing the compact extraoral evacuator suction tip would allow the compact extraoral evacuator suction tip to be placed in other locations besides directly above or on the upper portion of the bottom lip and pointing towards the entrance to a patient's oral cavity.

Having the option of using different sections for vent slot opening 10, the hollow cylindrical tube 11, wing-shaped collector 14, and cylindrical tube opening 12 could allow the compact extraoral suction passage tip to be less rigid, and therefore more flexible. Variations for cylindrical tube opening 12 and hollow cylindrical tube 11 could allow dentist offices to connect to different HVE, evacuator, and vacuum systems.

An autoclavable option could be used to be more environmentally and budget friendly. In embodiments in which various pieces are connected, these different pieces could be made of different materials to include High Density Polyethylene (HDPE), Polypropylene (PP) medical grade, Acetal Copolymer (POM-C), or other resilient of semi-resilient material. If made of various connected pieces, various pieces can be autoclavable or discarded between each use.

Changing ratios and dimensions of various portions of the compact extraoral suction passage tip could improve the ability to further reduce aerosols and splatter.

### CONCLUSION, RAMIFICATIONS, AND SCOPE

The compact extraoral suction passage tip can be used to reduce aerosols and other airborne contaminants while not significantly obstructing a dental professional's ability to perform procedures. The compact extraoral suction passage tip also reduces patient discomfort, as it will not be placed in or completely around the oral cavity.

Although the description above contains many details, these should not be construed as limiting the scope of the embodiments. Instead, these serve as examples of several embodiments. Thus, the scope of the embodiments should be determined by the claims and their legal equivalents, rather than the examples given.

What is claimed is:

1. A compact extraoral suction passage tip comprising:
a. a hollow cylindrical tube;
b. said hollow cylindrical tube having a means to be operably connected to an external vacuum-generating means on its proximal end;
c. a hollow wing-shaped collector connected to the distal end of the hollow cylindrical tube;
d. said hollow wing-shaped collector having a substantially circular cross-section at the proximal end that is connected to the hollow cylindrical tube and a vent slot opening on the distal end of the hollow wing-shaped collector;
e. said hollow wing-shaped collector having outer and inner surfaces that widen when traversing from the proximal to the distal end of said wing-shaped collector along a first axis that is normal to axial axis of said hollow cylindrical tube while becoming narrower along an axis normal to the axial axis and said first axis, with such narrowing occurring when traversing from the proximal to the distal end of said hollow wing-shaped collector;
f. the distal end of the hollow wing-shaped collector having a vent slot opening;
g. said hollow cylindrical tube and said wing-shaped collector composed of semi-resilient or resilient material.

2. The compact extraoral suction passage tip of claim 1 wherein the vent slot opening is substantially rectangular in shape.

3. The compact extraoral suction passage tip of claim 1 wherein the wing-shaped collector connects to the hollow cylindrical tube at an angle of approximately 45 degrees from the axial axis of the hollow cylindrical tube.

4. The compact extraoral suction passage tip of claim 1 wherein all items comprising the extraoral suction passage tip are made up of a continuous piece of material.

5. The compact extraoral suction passage tip of claim 1 wherein the items comprising the extraoral suction passage tip are configured to be separate pieces that can connect to one another.

6. The compact extraoral suction passage tip of claim 1 wherein a means to add friction to the hollow cylindrical tube or wing-shaped collector is added to help prevent movement.

7. The compact extraoral suction passage tip of claim 1 wherein the hollow cylindrical tube contains one or more semicircular beveled ridges to assist a patient to determine where to hold said compact extraoral suction passage tip.

8. The compact extraoral suction passage tip of claim 4 wherein the continuous material is selected from the group consisting of High Density Polyethylene (HDPE), Polypropylene (PP) medical grade, and Acetal Copolymer (POM-C).

9. The compact extraoral suction passage tip of claim 5 wherein said separate pieces are composed of material selected from the group consisting of High Density Polyethylene (HDPE), Polypropylene (PP) medical grade, and Acetal Copolymer (POM-C).

10. The compact extraoral suction passage tip of claim 4 wherein said continuous piece of material is autoclavable.

11. The compact extraoral suction passage tip of claim 5 wherein at least one said separate pieces are autoclavable.

12. The compact extraoral suction passage tip of claim 1 wherein the proximal end of the inner and outer surfaces of the hollow cylindrical tube are sized to fit within an external HVE valve.

13. A method of preventing the exchange of airborne and fluid substances, while providing visual and hand access by a practitioner to a patient's oral cavity, comprising:
a. connecting the proximal end of a hollow passage to an external vacuuming means,
b. providing said hollow passage to direct fluid flow to provide increasing amounts of suctioning strength when traversing toward a relatively narrow and wide vent slot opening at the distal end of the hollow passage when compared to the shape of the passage at the proximal end that was connected to the external vacuuming means, and
c. placing the vent slot opening on or around the upper portion of said patient's bottom lip with the vent slot opening pointing towards the entrance of the oral cavity.

14. The method of claim 13 wherein the hollow passage consists of material selected from the group consisting of High Density Polyethylene (HDPE), Polypropylene (PP) medical grade, and Acetal Copolymer (POM-C).

15. The method of claim 13 wherein the connection to the external vacuuming means is performed by sliding the hollow passage into an external HVE valve that is connected to an external central vacuum system via external HVE.

16. The method of claim 13 wherein the hollow passage comprises of a hollow cylindrical tube on its proximal end connected to a wing-shaped collector that has a vent slot opening on its distal end.

17. A means to suction particulates from a vent slot opening placed within, or, or near the upper portion of the bottom lip of a patient with the vent slot opening pointing towards the entrance of said patient's oral cavity, wherein the improvement comprises an operably connected external vacuum generating source on the proximal end and a vent slot opening at the distal end that is narrower along a first axis that is normal to the axial axis and wider along another axis that is normal to the axial axis and said first axis.

18. The improvement of claim 17 comprised of material selected from the group consisting of High Density Polyethylene (HDPE), Polypropylene (PP) medical grade, and Acetal Copolymer (POM-C).

19. The improvement of claim 17 wherein the connection to the external vacuum generating source means is performed by sliding the hollow passage on the proximal end of the improvement into an external HVE valve that is connected to an external central vacuum system via HVE.

20. The improvement of claim 17 comprised of a hollow cylindrical tube on its proximal end connected to a wing-shaped collector that has a vent slot opening on its distal end.
